(19) United States
(12) Patent Application Publication
(10) Pub. No.: US 2017/0166516 A1
(43) Pub. Date: Jun. 15, 2017

(54) STABLE POLYMORPH FORM B OF TAPENTADOL HYDROCHLORIDE

(71) Applicant: AZAD PHARMACEUTICAL INGREDIENTS AG, Schaffhausen (CH)

(72) Inventors: Dominique Anna BOCK, Mülheim an der Ruhr (DE); Thomas MAIER, Stockach (DE); Sven HAFERKAMP, Bochum (DE); Frank PORSTMANN, Stetten (CH)

(21) Appl. No.: 15/118,119

(22) PCT Filed: Feb. 10, 2015
(86) PCT No.: PCT/EP2015/052785

§ 371 Date: Aug. 11, 2016

(30) Foreign Application Priority Data
Feb. 12, 2014 (GB) 1402377.4

(51) Int. Cl. C07C 215/54; C07C 213/10

(52) U.S. Cl. CPC C07C 215/54; C07C 213/10; C07B 2200/13

(57) ABSTRACT

The present disclosure relates to Tapentadol Hydrochloride in the polymorphic crystalline Form B, which is substantially free of polymorphic Form A as well as essentially free of low alkyl carboxylic acids or esters of such acids. Furthermore, the present disclosure provides a process to produce this polymorphic Form B substantially free of Form A and its preparation and use for pharmaceutical compositions. This process as well as the specific crystalline form is uncommon, improved and industrially advantageous. Furthermore, the disclosure relates to pharmaceutical compositions and uses thereof.

### CROSS-REFERENCE TO RELATED APPLICATIONS

This application is a National Stage of International Application No. PCT/EP2015/052785, filed on Feb. 10, 2015, and published in English as WO 2015/121265 A1 on Aug. 20, 2015. This application claims the priority to Great Britain Application No. 1402377.4, filed on Feb. 12, 2014. The entire disclosures of the above applications are incorporated herein by reference.

### FIELD OF THE INVENTION

The present disclosure relates to Tapentadol Hydrochloride in the polymorphic crystalline Form B, which is substantially free of polymorphic Form A as well as essentially free of low alkyl carboxylic acids or esters of such acids. Furthermore, the present disclosure provides a process to produce this polymorphic Form B substantially free of Form A and its preparation and use for pharmaceutical compositions. This process as well as the specific crystalline form is uncommon, improved and industrially advantageous. Furthermore, the disclosure relates to pharmaceutical compositions and uses thereof.

### BACKGROUND

This section provides background information related to the present disclosure which is not necessarily prior art.

Tapentadol and its salt forms act, among other mode of actions, as opioid agonist and as noradrenalin uptake inhibitor. It has valuable pharmacological and therapeutic properties. For example, the drug acts centrally as analgesic. Tapentadol is chemically known as 3-[(1R,2R)-3-(dimethylamino)-1-ethyl-2-methylpropyl]phenol (formula I). As a hydrochloride-salt it is approved in pharmaceutical compositions.

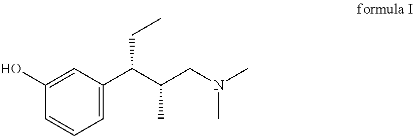 [SMILES: CC[C@@H](c1cccc(O)c1)[C@@H](C)CN(C)C]

Tapentadol Hydrochloride was the first time described by the patent family with the priority application DE 19 944 426 245 A1, of which priority date is Jul. 23, 1994. To this patent family belongs U.S. Pat. No. 6,071,970 A, U.S. RE39593 E1 and EP 693 475 A1, among other patents and applications. Tapentadol hydrochloride is example 25 in this patent family. However, stereo chemical descriptors have been mismatched in several documents by the authors of the patent applications. It was characterized as a compound with a melting point of 168-170° C. The hydrochloride salt of Tapentadol was obtained by a standard method, like e.g. adding a trimethylchlorsilan/water mixture as a source of hydrochloride to the free base Tapentadol. Consequently, the hydrochloride salt crystallized out.

In EP 1 612 203 A1, which claims priority of Jun. 28, 2004, two polymorphic forms of Tapentadol hydrochloride are described, named as Form A and Form B. In EP 1 612 203 A1 it is stated that Form B was obtained by the preparation methods of priority application DE 19 944 426 245 A1. Furthermore, polymorphic Form A is first time described in EP 1 612 203 A1. Several methods are disclosed to prepare either Form A or Form B. It is disclosed that the crystalline Form A has the same pharmacological activity than Form B, but is stable under ambient conditions, while Form B is descripted to be meta-stable only.

### SUMMARY

This section provides a general summary of the disclosure, and is not a comprehensive disclosure of its full scope or all of its features.

The phenomenon that a specific compound can occur in different crystalline forms is referred to as polymorphism. Different salts of an active pharmaceutical ingredient may possess different properties. Such variations in the properties of different salts may provide a basis for improving formulation. Different salts of an active pharmaceutical ingredient may also give rise to a variety of polymorphs or crystalline forms.

Polymorphism, the occurrence of different crystal forms, is a property of some molecules and molecular complexes. A single molecule, like Tapentadol and its salts, may give rise to a variety of polymorphs having distinct crystal structures and physical properties like melting point, thermal behaviors (e.g., measured by capillary melting point, thermo gravimetric analysis (TGA), or differential scanning calorimetry (DSC), as well as content of solvent in the polymorphic form, powder x-ray diffraction pattern (PXRD), infrared absorption and Raman fingerprints, and solid state NMR spectra). The differences in physical properties have been used to distinguish polymorphic forms. One or more of these techniques may be used to distinguish different polymorphic forms of a compound. These techniques may also be used to quantify the amount of one or more crystalline forms in a mixture.

The differences in the physical properties of different salts and polymorphic forms results from the orientation and intermolecular interactions of adjacent molecules or complexes in the bulk solid. Accordingly, polymorphs are distinct solids sharing the same molecular formula yet having distinct physical properties compared to other polymorphic forms of the same compound or complex.

The discovery of polymorphic forms of Tapentadol or its salt forms, specifically Tapentadol hydrochloride can provide new ways to improve the synthesis and the characteristics of Tapentadol as an active pharmaceutical ingredient.

In U.S. Pat. No. 8,288,592 B2 various crystalline and amorphous solid states of different salts of Tapentadol are disclosed.

WO 2012 051246 A1 discloses the description of several Tapentadol Hydrobromide polymorphic forms.

In WO 2013 111161 A1, which claims priority of Jan. 10, 2012, a stable polymorph B at ambient temperatures and several methods of preparing it are descripted. Stabilizing is achieved by adding additional components during crystallization resulting to a stability of the polymorphic Form B for several months. The additional components added during crystallization are low alkyl carboxylic acids or esters of such acids as stabilizer.

However, by these additional components, like formic acid and other low alkyl carboxylic acids, the obtained Tapentadol hydrochloride has additional components build in into the crystal structure of the solid form. It is very difficult to detect the additional components by methods like HPLC, because these additional components are not easily detectable by a UV/Vis detector attached to the analytical HPLC instrument. Additional components, even if they fall within the range of ICH guidelines, have always numerous disadvantages, like e.g. interaction with the main pharmacological action of the drug or increasing the undesired effects of the drug. Furthermore an alteration of the ADME behavior (adsorption, distribution, metabolism, excretion) is possible. These additional components might alter the absolute degree and the variability of the bioavailability, as well as the first pass metabolism, if they are co-absorbed. Furthermore the components might have an unpredictable influence of any transporter proteins, which are responsible for a potential active update of the API. Additional, undesired pharmacological effects are possible which may be caused by the impurities. In addition, taste, odor and overall consistence of the pharmaceutical product might be a drawback. It is more advantageous if these additional components are not present in a pharmaceutical preparation, also for regulatory reason.

Multiple processes for the preparation of Tapentadol Hydrochloride are published. e.g., WO 2008 012047 A1 and WO 2008 012046 A1 describe a synthesis via Mannich reaction including a diastereomeric salt resolution.

WO 2011 080736 A1 describes the preparation of Tapentadol through phenylpent-2-en amide

WO 2011 080756 A1 consists of the preparation of a cyano-intermediate as key intermediate towards Tapentadol preparation.

WO 2011 092719 A1 describes a method of Tapentadol preparation using the key intermediate (bromopropyl)methoxybenzene.

WO 2012 001571 A1 describes Tapentadol synthesis with a benzyl group as substituent at the amino function.

WO 2012 089177 A1 consists of the description of Tapentadol synthesis of a protected alkene acid as intermediate.

WO 2012 023147 A1 comprises the reaction of (dimethylamino)-2-methylpentan-3-one with anisole-Grignard.

WO 2012 038974 A1 comprises a Tapentadol preparation using 1-(3-hydroxyphenyl)propan-1-one with an appropriate Grignard reagent.

WO 2012 069004 A1 describes the Tapentadol synthesis using Methane sulfonyl esters.

WO 2012 103799 A1 describes the use of Evans auxiliary to generate key chiral intermediates for Tapentadol preparation.

It is always advantageous, if a stable polymorphic form is used for pharmaceutical application. Therefore, there is a need for a stable polymorphic Form B of Tapentadol Hydrochloride. It is also advantageous to have pharmaceutical active ingredients without additional chemical components as stabilizer, because these additional components increase the risk of additional impurities, which may have an overall impact on the suitability for the pharmaceutical use. Furthermore, there is improvement of shelf-life stability, dissolution rate, hygroscopicity, tableting properties, crystal morphology compact ability, density, hardness, powder flow ability, triboelectrical properties (electrostatic charging), chemical stability, optical stability, taste, odor, and regulatory unambiguousness (no mixture of different polymorphs in unclear proportions). It is therefore an object of the disclosure to provide a stable polymorphic Form B of Tapentadol Hydrochloride which is substantially free of polymorphic crystal Form A. In another aspect, it is an object of the invention to provide a method of production of a stable polymorphic Form B of Tapentadol Hydrochloride which is essentially free of low alkyl carboxylic acids or esters of such acids. Yet in another aspect of the disclosure a pharmaceutical composition is provide comprising a stable polymorphic Form B of Tapentadol Hydrochloride which is essentially free of low alkyl carboxylic acids or esters of such acids. In another aspect the disclosure relates to the use of a Tapentadol Hydrochloride in polymorphic crystal Form B which is substantially free of polymorphic crystal Form A for the preparation of a pharmaceutical composition.

### DEFINITIONS

Purity of the compositions is defined by methods of powder X-ray diffraction (PXRD). For the polymorphic crystal Form A the relevant peaks in the PXRD are at 2Theta 18.25°, 18.89°, 22.58° and 24.28° (limit+−0.3°). For the polymorphic crystal Form A the most relevant peaks in the PXRD are at 2Theta 18.89°, 22.58° and 24.28° (limit+−0.3°). In terms of the disclosure, the phrase “substantially free of polymorphic Form A” shall mean that the intensity of each of the most relevant peaks correlated with Form A are ≦1.5%, preferably ≦1% relative to the peak correlated with Form B at 2Theta 14.55° (+−0.3°).

Further substantially free of polymorphic Form A shall preferably be also understood that no visible peaks at 2Theta 18.25°, 18.89°, 22.58° and 24.28° can be found in a PXRD of the polymorphic Form B.

The term low alkyl carboxylic acid in the meaning of the disclosure refers to alkyl carboxylic acid comprising 1 to 4 carbon atoms, like e.g. formic acid, acetic acid, propionic acid, butyric acid, and isobutyric acid. Among others, esters of such low alky carboxylic acids are e.g. methyl formate, ethyl formate, propyl formate, butyl formate, isobutyl formate, methyl acetate, ethyl acetate, propyl acetate, butyl acetate, isobutyl acetate, methyl propionate, ethyl propionate, butyl propionate, isobutyl propionate, methyl butyrate, ethyl butyrate, propyl butyrate, butyl butyrate, amyl butyrate, and isopentyl butyrate.

The term essentially free of low alkyl carboxylic acids or esters of such acids shall mean that the overall concentration of the acids and/or esters in the composition is ≦3% by weight, preferably ≦2% by weight, more preferably ≦1% by weight, and most preferred ≦0.3% by weight.

When referring to ultrafine filtering in terms of the disclosure filtering of a solution and/or liquid by passing it through a filter means retaining particles >0.2 μm from passing, also referred to as ultrafine filter. An example for such filter means is e.g. a PTFE membrane filter having a pore size of 0.2 μm, like e.g. Whatman Rezist 13 filter.

### BRIEF DESCRIPTION OF THE DISCLOSURE

It was surprisingly found, that a stable polymorphic Form B is obtained when there are no crystal seeds/nucleation centers of polymorphic Form A present. Furthermore, surprisingly it was found that heating of solid Tapentadol Hydrochloride with or without amorphous portions results in a purification of polymorphic Form B in such manner that the resulting solid is substantially free of polymorphic Form A. Furthermore, it was found that in the absence of polymorphic from A no transformation of polymorphic Form B into polymorphic Form A occurs, which renders Form B to be a stable polymorphic form.

A Tapentadol Hydrochloride in stable polymorphic crystal Form B which is substantially free of polymorphic crystal Form A is characterized in that the composition exhibits a PXRD pattern in which the intensity of peaks at 2Theta 18.89°, 22.58° and 24.28° (+−0.3°) are each ≦1.5%, preferably ≦1% relative to the peak at 2Theta 14.55° (+−0.3°), more specifically at 2Theta 18.25°, 18.89°, 22.58° and 24.28° (+−0.3°) are each ≦1.5%, preferably ≦1% relative to the peak at 2Theta 14.55° (+−0.3°).

While the polymorphic crystal Form B of Tapentadol Hydrochloride is already known from the state of the art, like e.g. EP 1 612 203 A1 or WO 2013 111161 A1, it is novel to provide such polymorphic crystal Form B which is substantially free of polymorphic crystal Form A and also being substantially free of low alkyl carboxylic acids and/or esters of such acids. While the presence of low alkyl carboxylic acid and/or esters of such acids may be difficult to prove by common analytic methods, the Tapentadol Hydrochloride known from the state of the art has a characteristic odor indication the presence of such acids and esters. However, surprisingly it was found that the absence of said low alkyl carboxylic acids and/or esters of such acids can be proven by PXRD, too.

Low alkyl carboxylic acids and/or esters of such acids are deemed to be encased or incorporated into the crystal structure of the composition, thereby causing a disturbance of the symmetry of the crystal structure. Since the low alkyl carboxylic acids and/or esters of such low alkyl carboxylic acids are not distributed evenly within the crystal structure they cause a fuzziness of the PXRD pattern which results in a broaden Full-Width-at-Half-Maximum (FWHM) of significant peaks.

Full width at half maximum (FWHM) is an expression of the extent of a function, given by the difference between the two extreme values of the independent variable at which the dependent variable is equal to half of its maximum value. FWHM is typically applied to such phenomena as the resolution of spectrometers.

For example, if the considered function is the normal distribution of the form

$$f ( x ) = \frac{1}{σ \sqrt{2 π}} exp [ - \frac{{( x - {x}_{0} )}^{2}}{2 {σ}^{2}} ]$$

where σ is the standard deviation and x₀ can be any value (the width of the function does not depend on translation), then the relationship between FWHM and the standard deviation is

FWHM=2\sqrt{2 ln 2}σ≈2.355 σ.

For the inventive Tapentadol Hydrochloride in stable polymorphic crystal Form B which is essentially free of low alkyl carboxylic acids and/or esters of such low alkyl carboxylic acids it was found that the FWHM value for the most relevant peaks is significantly ≦0.2, preferably ≦0.18, and even more preferred ≦0.16.

More specifically, it was found that a PXRD pattern of a Tapentadol Hydrochloride in stable polymorphic crystal Form B which is essentially free of low alkyl carboxylic acids and/or esters of such low alkyl carboxylic acids shows a relevant peak at 2Theta 17.994° (±0.3°) having a FWHM of ≦0.091. Preferably said pattern shows additionally another relevant peak at 2Theta 19.582° (±0.3°) having a FWHM of ≦0.105. Even more preferred such pattern shows a third relevant peak at 2Theta 21.979° (±0.3°) having a FWHM of ≦0.111, and in a most preferred embodiment of the disclosure the PXRD pattern shows a fourth relevant peak at 2Theta 28.174° (±0.3°) having a FWHM of ≦0.153.

A comparison of the PXRD pattern of a Tapentadol Hydrochloride in polymorphic crystal structure Form B as known from the state of the art with the inventive Tapentadol Hydrochloride in polymorphic crystal Form B which is substantially free of low alkyl carboxylic acids and/or esters of such low alkyl carboxylic acids results in that the FWHM values of the relevant peaks in the pattern of the composition known from the state of the art, like e.g. WO 2013 111161 A1, are at least 2 times higher than the FWHM values of the inventive composition. Accordingly, it can be assumed that the inventive Tapentadol Hydrochloride in stable crystal Form B comprises significant lower concentration of low alkyl carboxylic acids and/or esters of such low alkyl carboxylic acids which disturb the symmetry of the crystal structure. Moreover, a comparison of the PXRD pattern of an inventive Tapentadol Hydrochloride in stable polymorphic crystal structure Form B with mono-crystal structure date shows, that the FWHM values of the inventive composition are in the same order as the FWHM values of the mono-crystal X-ray structure data. Accordingly, it can be assumed that the inventive Tapentadol Hydrochloride in stable polymorphic crystal structure From B is essentially free of any low alkyl carboxylic acids and/or esters of such low alkyl carboxylic acids.

Accordingly, a process of preparing Tapentadol Hydrochloride substantially in a stable polymorphic pure Form B is provided, wherein the process comprises the removal of nucleation centers and subsequent crystallization.

Surprisingly it was found that by such a process Tapentadol Hydrochloride can be provided which is substantially in a stable polymorphic crystal Form B and which is essentially free of low alkyl carboxylic acids and/or ester of such low alkyl carboxylic acids.

According to a preferred embodiment, a process of preparing Tapentadol Hydrochloride substantially in a stable polymorphic pure Form B is provided, wherein the process comprises the step of
filtering a solution of Tapentadol Hydrochloride with an ultrafine filter; and
crystallizing from filtrate a Tapentadol Hydrochloride.

In another embodiment of the disclosure, the process crystallization is performed at elevated temperatures, preferably ≧50° C., more preferably ≧52° C. It was found that crystallization at elevated temperature beneficially results in low concentration of Tapentadol Hydrochloride polymorphic crystal Form A.

In another preferred embodiment, wherein the process comprises the step of refluxing a solution of Tapentadol Hydrochloride at temperatures between 55-90° C. for 2 hours to 300 hours, preferably for at least 5 hours, more preferably for at least 24 hours. Like for the crystallization at elevated temperature it was found that beneficially the refluxing for said time results in low concentration of Tapentadol Hydrochloride polymorphic crystal Form A. Refluxing a solution of Tapentadol Hydrochloride for a period of time longer than 2 hour results in a purification of the later obtained solid Tapentadol Hydrochloride. After said refluxing time the resulting polymorphic stable Form B is substantially free of any seed of polymorphic Form A.

In another preferred embodiment of the disclosure, the solution of Tapentadol Hydrochloride comprises as solvent at least one solvent of the group consisting of alcohols, preferentially C1-C4 alcohols, ethers, esters, hydrocarbons or halogenated hydrocarbons, nitriles, or ketones, e.g., but not limited to, tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone as well as mixtures thereof.

Yet in another preferred embodiment of the disclosure, the process comprises the step of dissolving Tapentadol as free base in at least one solvent of the group consisting of tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone and adding hydrochloric acid to the solution prior to refluxing the solution. By addition of hydrochloric acid the free base of Tapentadol is converted into the hydrochloric salt species. Hydrochloric acid may be added in form of a gas and/or a solution, like e.g. an aqueous solution. In an even more preferred embodiment, the adding of hydrochloric acid is performed after the step of ultrafine filtering of the solution.

Preferably, during refluxing further Tapentadol is added to the solution, preferably up to the maximum solubility. By this the later crystallization is eased significantly.

According to another preferred embodiment of the disclosure, after ultrafine filtration the filtrate is seeded with a seed crystal of pure polymorphic Form B of a Tapentadol Hydrochloride. If the solution is refluxed, such seeding can be performed either prior or after refluxing of the solution while seeding prior to refluxing is preferred.

In a preferred embodiment of the disclosure the process comprises the steps of
dissolving Tapentadol as free base in at least one solvent of the group consisting of tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone;
ultrafine filtering of the solution of the free base;
seeding the filtrate with a seed crystal of pure polymorphic Form B of a Tapentadol Hydrochloride;
adding hydrochloric acid to the seeded filtrate; and
crystallizing from the seeded filtrate a Tapentadol Hydrochloride.

According to another embodiment of the disclosure, the process comprises the steps of
dissolving Tapentadol as free base in at least one solvent of the group consisting of tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone;
ultrafine filtering of the solution of the free base;
seeding the filtrate with a seed crystal of pure polymorphic Form B of a Tapentadol Hydrochloride;
adding hydrochloric acid to the seeded filtrate;
refluxing the solution at temperatures between 55-90° C. for 2 hours to 300 hours, preferably for at least 5 hours, more preferably for at least 24 hours; and
crystallizing from the seeded filtrate a Tapentadol Hydrochloride.

According to another embodiment of the disclosure, the process comprises the steps of
dissolving Tapentadol as free base in the at least one solvent of the group consisting of tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone;
ultrafine filtering of the solution of the free base;
seeding the filtrate with a seed crystal of pure polymorphic Form B of a Tapentadol Hydrochloride;
adding hydrochloric acid to the seeded filtrate;
refluxing the solution at temperatures between 55-90° C. for 2 hours to 300 hours, preferably for at least 5 hours, more preferably for at least 24 hours; and
crystallizing from the seeded filtrate a Tapentadol Hydrochloride at elevated temperatures, preferably ≧50° C., more preferably ≧52° C.

According to another embodiment of the disclosure, the process comprises the steps of
dissolving Tapentadol Hydrochloride in the at least one solvent of the group consisting of tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone;
ultrafine filtering of the solution of the Tapentadol Hydrochloride;
refluxing the solution at temperatures between 55-90° C. for 2 hours to 240 hours, preferably for at least 5 hours, more preferably for at least 24 hours; and
crystallizing from the filtrate a Tapentadol Hydrochloride at elevated temperatures, preferably ≧50° C., more preferably ≧52° C.

According to another embodiment of the disclosure, the process comprises the steps of
dissolving Tapentadol Hydrochloride in the at least one solvent of the group consisting of tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone;
ultrafine filtering of the solution of the Tapentadol Hydrochloride;
seeding the filtrate with a seed crystal of pure polymorphic Form B of a Tapentadol Hydrochloride;
refluxing the solution at temperatures between 55-90° C. for 2 hours to 300 hours, preferably for at least 5 hours, more preferably for at least 24 hours; and
crystallizing from the seeded filtrate a Tapentadol Hydrochloride at elevated temperatures, preferably ≧50° C., more preferably ≧52° C.

Furthermore, the disclosure relates to the use of a Tapentadol Hydrochloride in stable polymorphic crystal Form B which is substantially free of polymorphic crystal Form A, characterized in that the composition exhibits a PXRD pattern in which the intensity of peaks at 2Theta 18.25°, 18.89°, 22.58° and 24.28° (+−0.3°) are each ≦1.5%, preferably ≦1% relative to the peak at 2Theta 14.55° (+−0.3°), more specifically at 2Theta 18.89°, 22.58° and 24.28° (+−0.3°) are each ≦1.5%, preferably ≦1% relative to the peak at 2Theta 14.55° (+−0.3°) for the preparation of a pharmaceutical composition.

In a preferred embodiment the disclosure relates to the use of Tapentadol Hydrochloride exhibiting a PXRD pattern having peaks at 2Theta 17.99°, 19.58° and 21.99° (+−0.3°), wherein the Full-Width-at-Half-Maximum (FWHM) of each of these peaks is ≦0.2, preferably ≦0.16. Even more preferred the disclosure relates to the use of Tapentadol Hydrochloride exhibiting a PXRD pattern having peaks at 2Theta 17.99°, 19.58° and 21.99° (+−0.3°), and a peak at 2Theta 28.17° (+−0.3°), wherein the Full-Width-at-Half-Maximum (FWHM) of each of these peaks is ≦0.2, preferably ≦0.16.

Preferably, the use according to the disclosure encompasses the manufacturing of a finished dosage form which is substantially free of Tapentadol Hydrochloride Form A. Said finished dosage is preferably in the form of a solid dosage and most preferred in the solid dosage is a tablet or capsule or any other solid or semi-sold formulation for oral application.

### BRIEF DESCRIPTION OF THE DRAWINGS

The drawings described herein are for illustrative purposes only of selected embodiments and not all possible implementations, and are not intended to limit the scope of the present disclosure.

FIGS. 1 to 5 show PXRDs of Polymorphic Form A as well as B and/or mixtures thereof.

### DETAILED DESCRIPTION

Example embodiments will now be described more fully with reference to the accompanying drawings.

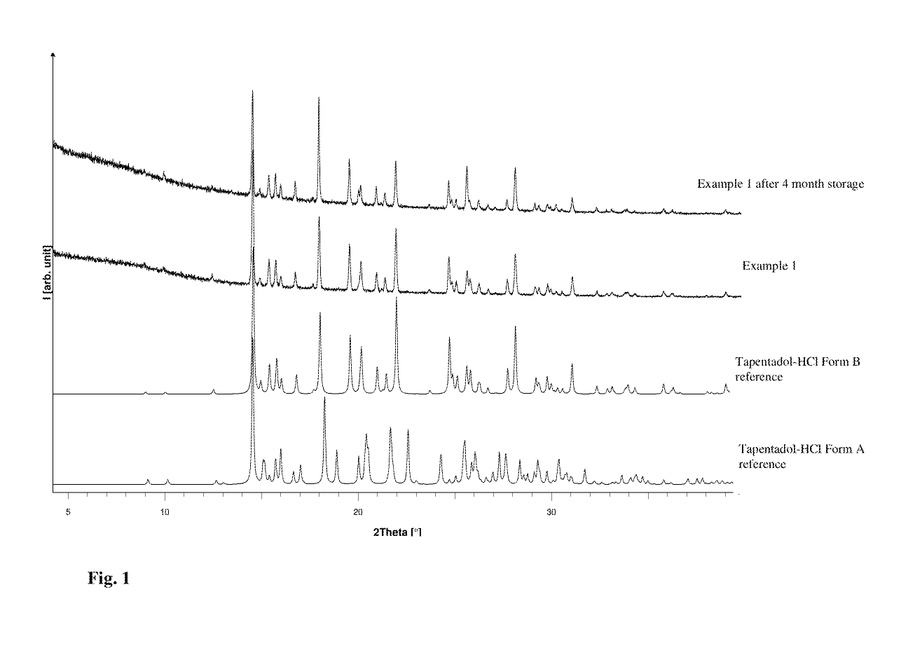

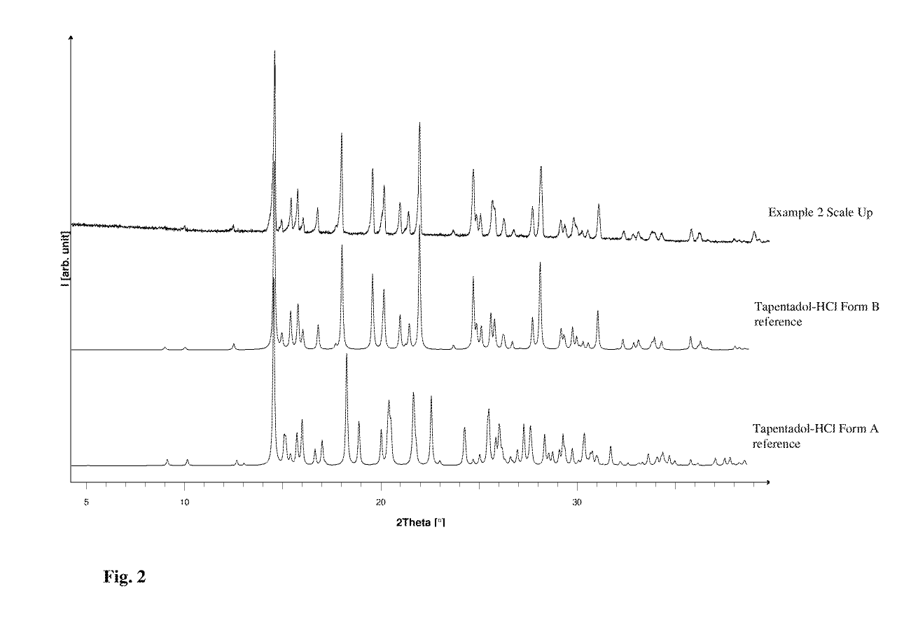

In FIG. 1 the PXRD pattern of a stability sample experiment 1 are shown as well as PXRD pattern of Tapentadol hydrochloride in polymorphic crystal structure A and in polymorphic crystal structure B. The upper most pattern results from a sample (experiment 1) after a 4 month stability test at 27° C. and 42% RH (relative humidity). The second of top pattern results from the initial sample prior to the stability testing. The second last pattern represents the mono-crystal structure of pure Tapentadol Hydrochloride in polymorphic crystal Form B. The last pattern represents the mono-crystal structure of pure Tapentadol Hydrochloride in polymorphic crystal Form A. What can be derivate from figure I is that the inventive Tapentadol Hydrochloride in stable polymorphic crystal Form B does not show any tendency to disintegrate or to transform to polymorphic crystal Form A. Furthermore, the comparison of the PXRD pattern of the inventive Tapentadol Hydrochloride in stable polymorphic crystal Form B with the mono-crystal structure of crystal Form B of Tapentadol Hydrochloride let derivate that the inventive composition is essentially free of low alkyl carboxylic acids and/or esters of such low alkyl carboxylic acids. FIG. 2 shows in the upper pattern PXRD pattern of Tapentadol hydrochloride Form B from a scale up experiment. The second last pattern represents the mono-crystal structure of pure Tapentadol Hydrochloride in polymorphic crystal Form B. The last pattern represents the mono-crystal structure of pure Tapentadol Hydrochloride in polymorphic crystal Form A.

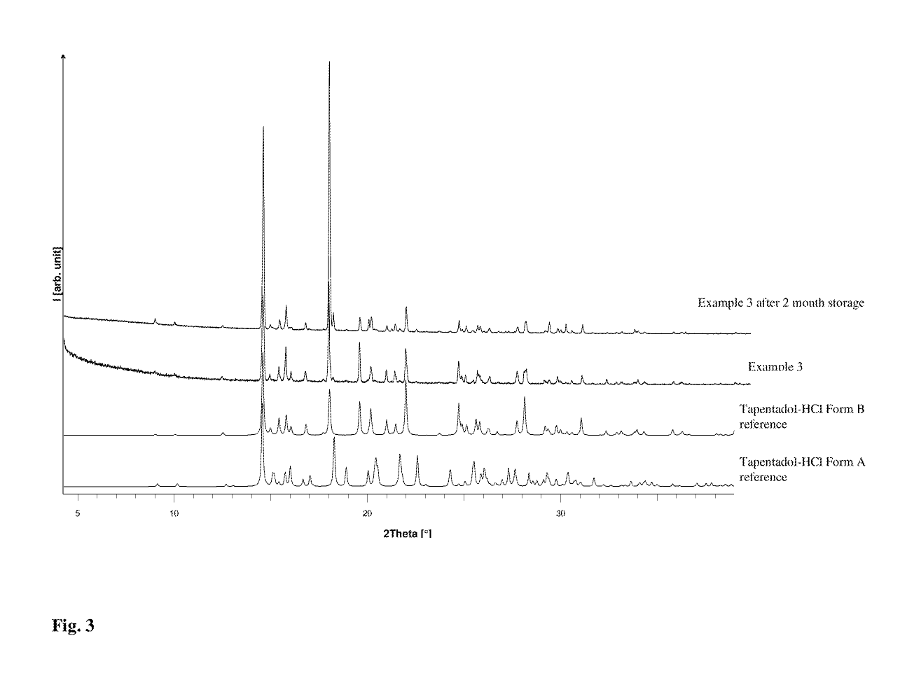

FIG. 3 shows Tapentadol Hydrochloride PXRD pattern of polymorphic Form A as well as B. The exemplified experimental polymorphic Form B in this case comprises some impurities of polymorphic Form A. After 2 month under ambient temperature storage conditions the amount of polymorphic Form A obviously increased. This is in contrast to behavior of pure Form B as exemplified in figure I. After several months of storage under ambient conditions polymorphic pure Form B without any seeds of Form A showed no transformation to polymorph A.

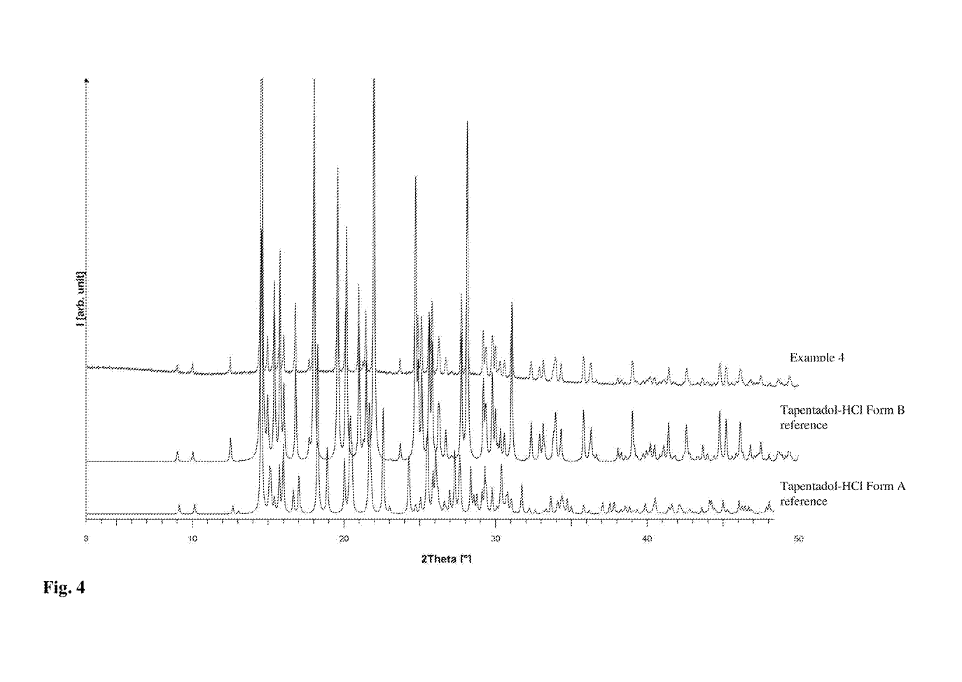

FIG. 4 shows Tapentadol Hydrochloride PXRD pattern of polymorphic Form A as well as B. Form B results the from a 1,4-Dioxan solution as explained in detail in example 4.

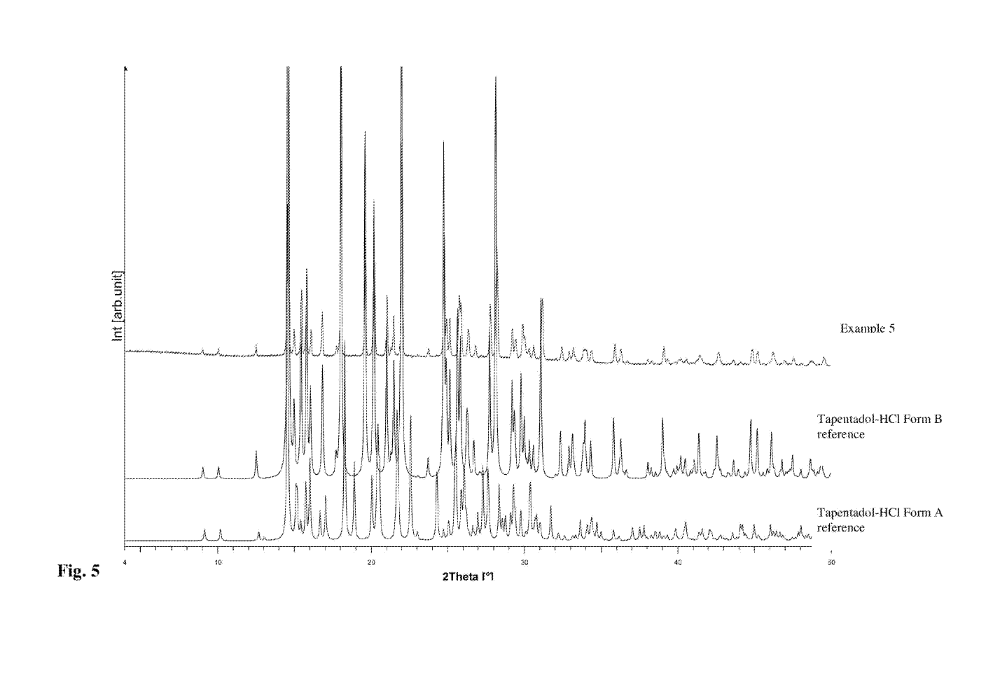

FIG. 5 shows Tapentadol Hydrochloride PXRD pattern of polymorphic Form A as well as B. Form B results the from a 2-methyl-tetrahydrofuran solution as explained in detail in example 5.

### EXPERIMENTAL EXAMPLES

Tapentadol as free base is dissolved in a mixture of solvents. Hydrochloric acid is added and the reaction mixture is heated under reflux temperature. Alternatively, Tapentadol hydrochloride can be dissolved in a mixture of solvents.

Further Tapentadol Hydrochloride might be added during refluxing, until a solid appears. The resulting hot dispersion is filtered with a preheated Whatman filter (0.2 μm) and the resulting solution is heated for 1 week at reflux temperature. The solvent is evaporated at temperatures above 60° C. and the resulting solid is isolated. This solid consist of polymorphic Form B substantially free of polymorph A.

Stable Form B without any additional stabilizing components is advantageous in terms of technical feasibility at ambient or elevated temperatures during processing into pharmaceutical compositions and storage of the API and pharmaceutical compositions. Stable Form B might have a lower hygroscopicity and improved flow behavior.

For Powder X-Ray Diffraction (PXRD) the samples were placed onto a Si-wafer (Bragg-Brentano) or into a standard glass capillary (Ø=0.7 mm) (Example 2). The measurements were performed at room temperature with a D8 Bruker Advance Diffractometer (Cu-Kα1=1.54059 Å, Johansson primary beam monochromator, position sensitive detector) in transmission mode with rotation of the sample.

### Example 1

Tapentadol Hydrochloride is dissolved in chloroform, and then heated to boiling point under reflux. Tapentadol Hydrochloride Form A and B was added under stirring until sediment is visible. The resulting dispersion (total mass ˜30 mg in 13 mL chloroform) was filtered through a preheated Buchner funnel (Whatman filter paper, white) and the solution stirred for one week at boiling temperature under reflux. The solvent is distilled off at atmospheric pressure gently to dryness. The solid phase afterwards was characterized by X-ray powder diffraction.

### Example 2 Scale Up

Tapentadol Hydrochloride is dissolved in chloroform, and then heated to boiling point under reflux. Tapentadol Hydrochloride Form A and B was added under stirring until sediment is visible. The resulting dispersion (total mass ˜455 mg in 200 mL chloroform) was filtered through a preheated Buchner funnel (Whatman filter paper, white) and the solution stirred for one week at boiling temperature under reflux. The solvent is distilled off at atmospheric pressure gently to dryness. The solid phase afterwards was characterized by X-ray powder diffraction.

### Example 3

The investigated sample shows Form B and traces of Form A of Tapentadol Hydrochloride after preparation. After two months of storage an increase of Form A in Form B was observed.

While the disclosure has been illustrated and described in detail in the drawings and foregoing description, such illustration and description are to be considered illustrative or exemplary and not restrictive; the disclosure is not limited to the disclosed embodiments. Other variations to the disclosed embodiments can be understood and effected by those skilled in the art in practicing the claimed disclosure, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Example 4

A solution of 360 mg Tapentadol free base in 5 ml 1,4-Dioxan are cooled to 15° C. To this solution 5 mg of Tapentadol HCl Form B are added under vigorous stirring. 0.5 ml of a 4 N HCl in Dioxan solution are added dropwise with a syringe within ca. 3 min while the internal temperature did not exceed 20° C. Immediately a colorless precipitate is formed. After completion of the HCl addition the suspension is stirred for additional 5 min at ambient temperature before the solid is filtered off. The wet product is dried in high vacuum yielding into pure Form B as per pXRD measurement. Yield: 300 mg (71%).

### Example 5

A solution of 0.75 g of Tapentadol free base 40 ml of 2-Methyl-tetrahydrofuran (2-MeTHF, water content <0.05%) was stirred in a three necked 250 ml round bottom flask equipped with a reflux condenser, CaCl₂-tube, thermometer and a aeration tube for introduction of the gas. The IT was kept at ambient temperature (23° C.). 20 mg Tapentadol HCl Form B were added and the suspension was vigorously stirred, HCl gas was bubbled directly into the solution. The precipitation of a very fine solid started immediately with the first gas flow. The internal temperature (IT) raised to approx. 29° C. and spontaneously a yellowish/colorless solid precipitates. The gas addition was stopped after 20 seconds. The apparatus was flushed for two minutes with nitrogen before the filtration of the solid was started. The solid collected by filtration. The filter cake was washed with 5 ml of 2-MeTHF at ambient temperature. The solid was sucked dry on the filter and the wet product submitted to high vacuum drying in a round bottom flask at 75° C. for 12-15 h, delivering pure form B as per pXRD measurement. Yield: 0.89 g (ca. 100%).

### Example 6

0.9 g of Tapentadol free base was dissolved in 45 ml of Methyl-isobutyl-ketone (MIBK) in a three necked round bottom flask equipped with a reflux condenser, CaCl₂-tube, thermometer and an aeration tube for introduction of the gas. The IT was kept at ambient temperature. 25 mg Tapentadol HCl Form B were added and the suspension was vigorously stirred. HCl gas was bubbled through the solution. The precipitation of a very fine solid started immediately with the first gas flow. The temperature was raised to approx. 29° C. and spontaneously a yellowish/colorless solid precipitates. The gas addition was stopped after 30 seconds. The apparatus was flushed for two minutes with nitrogen before the filtration of the solid was started. The solid collected by filtration. The filtrate was washed with 5 ml of MIBK at ambient temperature. The solid was sucked dry on the filter and was submitted to high vacuum drying at 75° C. for 17 h, delivering pure form B as per pXRD measurement. Yield: 0.85 g. (99%).

### Example 7

A solution of Tapentadol HCl salt (6.0 g) in methyl-isobutyl-carbinol (MIBC) (300 mL) was refluxed for 30 min to give a clear solution. Additional 90 mL of MIBC was added into the solution, then about 60 mL of MIBC was distilled off (to remove H₂O) and the clear solution was refluxed for 3 days. About 250 mL of MIBC was distilled off to give a slurry, which was cooled to ambient temperature over 3 h. The solid was collected and dried at 80° C. under vacuum to give 4.3 g, delivering pure Form B as per pXRD measurement. Yield: 4.3 g (60%).

1. A Tapentadol Hydrochloride in stable polymorphic crystal Form B which is substantially free of polymorphic crystal Form A, wherein the composition exhibits a PXRD pattern in which the intensity of peaks at 2Theta 18.89°, 22.58° and 24.28° (+−0.3°) are each ≦1.5%, preferably ≦1% relative to the peak at 2Theta 14.55° (+−0.3°), more specifically at 2Theta 18.25°, 18.89°, 22.58° and 24.28° (+−0.3°) are each ≦1.5%, preferably ≦1% relative to the peak at 2Theta 14.55° (+−0.3°).

2. The Tapentadol Hydrochloride according to claim 1, wherein the composition exhibits a PXRD pattern having peaks at 2Theta 17.99°, 19.58° and 21.99° (+−0.3°), wherein the Full-Width-at-Half-Maximum (FWHM) of each of these peaks is ≦0.2, preferably ≦0.16.

3. The Tapentadol Hydrochloride according to claim 2, wherein the composition exhibits a PXRD pattern furthermore has a peak at 2Theta 28.17° (+−0.3°), wherein the Full-Width-at-Half-Maximum (FWHM) of the peak is ≦0.2, preferably ≦0.16.

4. A process of preparing Tapentadol Hydrochloride substantially in a stable polymorphic pure Form B, wherein the process comprises the removal of nucleation centers and subsequent crystallization.

5. A process of preparing Tapentadol Hydrochloride substantially in a stable polymorphic pure Form B, wherein the process comprises the step of filtering a solution of Tapentadol Hydrochloride with an ultrafine filter; and crystallizing from the seeded filtrate a Tapentadol Hydrochloride.

6. The process according to claim 4, wherein the crystallization is performed at elevated temperatures, preferably ≧50° C., more preferably ≧52° C.

7. The process according to claim 4, wherein the process comprises the step of refluxing a solution of Tapentadol Hydrochloride at Temperatures between 55-90° C. for 2 hours to 300 hours, preferably for at least 5 hours, more preferably for at least 24 hours.

8. The process according to claim 6, wherein the solution comprises as solvent at least one solvent of the group consisting of alcohols, ethers, esters, hydrocarbons or halogenated hydrocarbons, nitriles, or ketones, as well as mixtures thereof.

9. The process according to claim 6, wherein the solution comprises as solvent at least one solvent of the group consisting of tetrahydrofuran, chloroform, dichloromethane, 3-methyl-1-butanole, methanol, ethanol, isopropanol, butanol, toluene, p-xylene, acetonitrile, 2-methyl-tetrahydrofuran, 1,4-dioxane, methyl isobutyl ketone, 2-methyl-tetrahydrofuran, 1,4-dioxane, methyl isobutyl ketone, methyl isobutyl carbinol, 2-methoxy-2-methylpropane (MTBE), ethylacetate, acetone and 2-butanone.

10. The process according to claim 9, comprising the step of dissolving Tapentadol as free base in the at least one solvent and adding hydrochloric acid to the solution prior to refluxing the solution.

11. The process according to claim 6, wherein during refluxing further Tapentadol is added to the solution, preferably up to the maximum solubility.

12. The process according to claim 5, wherein after filtering the filtrate is seeded with seed crystal of pure polymorphic Form B of a Tapentadol Hydrochloride.

13. Use of a Tapentadol Hydrochloride according to claim 1 for the preparation of a pharmaceutical composition.

14. The use of a Tapentadol Hydrochloride according to claim 11 for the manufacturing of a finished dosage form which is substantially free of Tapentadol Hydrochloride Form A.

15. The use of a Tapentadol Hydrochloride according to claim 12, wherein the finished dosage form is a solid dosage.

16. The use of a Tapentadol Hydrochloride according to claim 13, wherein the solid dosage form is a tablet.
